(19) United States
(12) Patent Application Publication
(10) Pub. No.: US 2010/0113831 A1
(43) Pub. Date: May 6, 2010

(54) Highly Pure Crystalline Benzphetamine Hydrochloride and Processes for Preparing

(75) Inventors: Keith G. Tomazi, Florissant, MO (US); Gary A. Nichols, Wildwood, MO (US); Michelle R. Menze, St.Luis, MO (US); Dennis J. Kalota, Fenton, MO (US)

Correspondence Address: Mallinckrodt Inc., 675 McDonnell Boulevard, HAZELWOOD, MO, 63042, US

(73) Assignee: MALLINCKRODT INC., St. Louis, MO (US)

(21) Appl. No.: 12/444,597

(22) PCT Filed: Oct. 17, 2006
(86) PCT No.: PCT/US2006/040415

§ 371 Date: Apr. 7, 2009

(51) Int. Cl. C07C 211/27

(52) U.S. Cl. 564/381

(57) ABSTRACT

A highly pure crystalline form of benzphetamine hydrochloride and methods for the purification and crystallization of benzphetamine hydrochloride in high yield are disclosed.

### FIELD OF THE INVENTION

The present invention relates to benzphetamine hydrochloride. More particularly, it relates to highly pure crystalline benzphetamine hydrochloride and processes for preparing crystalline benzphetamine hydrochloride.

### BACKGROUND OF THE INVENTION

Benzphetamine hydrochloride, an amphetamine derivative which is an anorectic (also known as “anorexigenic” or appetite suppressant), has found use in a variety of applications. The drug was first synthesized by Heinzelman et al. (U.S. Pat. No. 2,789,138) who found it to be a superior bronchodilator. Heinzelman et al. disclosed a process for the preparation of benzphetamine hydrochloride by the benzylation of d-desoxyephedrine (dextro-methamphetamine) in inert solvent. The dextro-benzphetamine product was dissolved in ethyl acetate and reacted with ethanolic hydrogen chloride to yield the benzphetamine hydrochloride salt. As reported by Heinzelman et al., the benzphetamine base is a colorless, water insoluble liquid. The water soluble hydrochloride salt was reported to have a melting temperature of 129° C. to 130° C. Current literature, however, indicates that benzphetamine hydrochloride has a melting point between 152° C. to 153° C. (See the Merck Index, 10^{th} ed.).

Today, benzphetamine hydrochloride is marketed as DIDREX®, a weight loss product and anti-obesity preparation, which acts mainly by suppressing the appetite. The drug has been shown efficacious as part of formulations for decreasing appetite and for the long term management of obesity. See U.S. Pat. No. 5,543,405 issued to Keown et al., U.S. Pat. No. 5,019,594 issued to Wurtman et al., and U.S. Pat. No. 4,895,845 issued to Seed. Benzphetamine hydrochloride has even been used in formulations designed to alleviate withdrawal symptoms due to the cessation of tobacco use. See U.S. Pat. Nos. 6,166,032 and 5,900,418, both issued to Viner.

The discovery of a high purity crystalline benzphetamine hydrochloride provides an opportunity to improve the performance characteristics of a pharmaceutical product.

Further, among the problems associated with the successful commercial development of formulations incorporating benzphetamine hydrochloride, the synthesis of the compound typically yields an unsolidified oily substance of low purity. Accordingly, a post-synthesis method for the purification and crystallization of benzphetamine hydrochloride is needed which yields a higher purity product.

### SUMMARY OF THE INVENTION

Among the various aspects of the present invention may be noted the provision of very high purity solid crystals of benzphetamine salt and a method for the purification and crystallization of benzphetamine salt in high yield.

In one aspect of the present invention, the crystalline form of benzphetamine hydrochloride has a purity of at least about 95% to about 100% by weight. In another aspect, the crystalline form of benzphetamine hydrochloride includes no more than 0.15% of any single impurity.

In yet another aspect of the present invention, the crystalline form of benzphetamine hydrochloride has a melting point between about 151° C. and about 158° C.

In still yet another aspect of the present invention, the highly pure crystalline form of benzphetamine hydrochloride is prepared by dissolving benzphetamine hydrochloride in a solvent system including an organic solvent and an organic modifier to form a solution, heating the solution to a temperature sufficient to dissolve the benzphetamine hydrochloride in the solvent system, and cooling the solution to a temperature sufficient to precipitate benzphetamine hydrochloride into the highly pure crystalline form of benzphetamine hydrochloride.

In another aspect of the present invention, the highly pure crystalline form of benzphetamine hydrochloride is prepared by dispersing benzphetamine hydrochloride in a liquid medium in which benzphetamine hydrochloride is essentially insoluble to form a biphasic mixture, heating the biphasic mixture to a temperature between about 40° C. and about 95° C., and cooling the biphasic mixture to precipitate the highly pure crystalline form of benzphetamine hydrochloride.

Other objects and aspects of the invention will be, in part, pointed out and, in part, apparent hereinafter.

### BRIEF DESCRIPTION OF THE DRAWINGS

FIG. 1 is a differential scanning calorimetry thermogram of benzphetamine hydrochloride.

FIG. 2 is a characteristic powder X-ray diffraction pattern of benzphetamine hydrochloride.

### DETAILED DESCRIPTION OF THE PREFERRED EMBODIMENT(S)

Benzphetamine hydrochloride is typically present as an oily liquid. Thus, the purification and crystallization processes of the present invention advantageously yield high purity solid benzphetamine hydrochloride. The purification and crystallization processes of the present invention yield benzphetamine hydrochloride having a purity of at least about 95% to about 100% in about 60% to about 80% overall yield. Further, the final product contains no more than 0.15% of any single impurity and residual solvents are removed such that they are present at levels below permitted safety standards. Finally, the crystalline benzphetamine purified according to the processes of the present invention have melting points between about 151° C. and about 158° C., more preferably between about 155° C. and about 157° C., which indicates its high degree of purity.

The highly pure crystalline form of benzphetamine hydrochloride of the present invention has been characterized by powder X-ray diffraction (“PXRD”) analysis and thermal methods including differential scanning calorimetry (“DSC”).

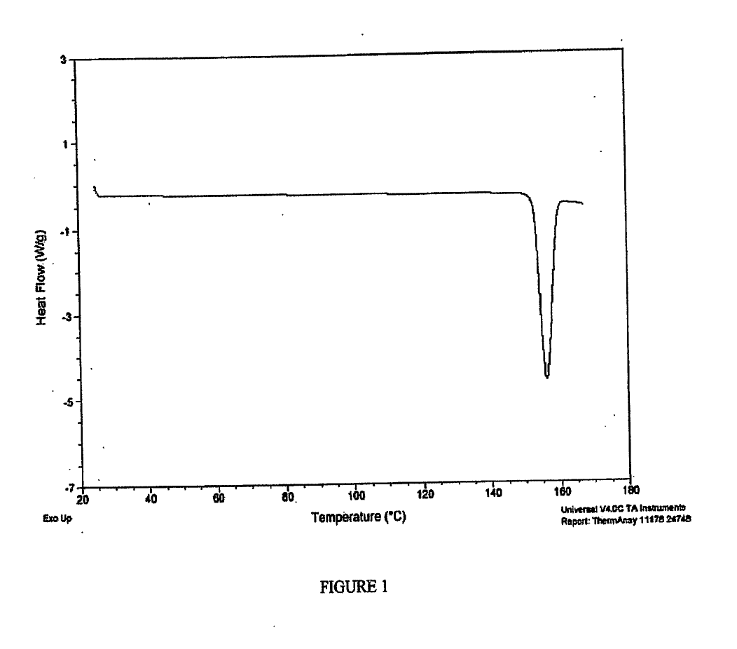

Referring to FIG. 1, the DSC thermogram of benzphetamine hydrochloride demonstrates the thermal stability of this crystalline form. FIG. 1 possesses an endothermic transition (melting/decomposition related) with an onset of approximately 150-155° C. DSC was performed using TA Instruments Q100. A portion of each sample was weighed into a crimped aluminum sample pan and sealed, and the sample was heated from 25° C. to 175° C. at a rate of 5° C./minute. Each sample exhibited a single endothermic transition as measured by DSC, which is associated with melting/decomposition of the sample.

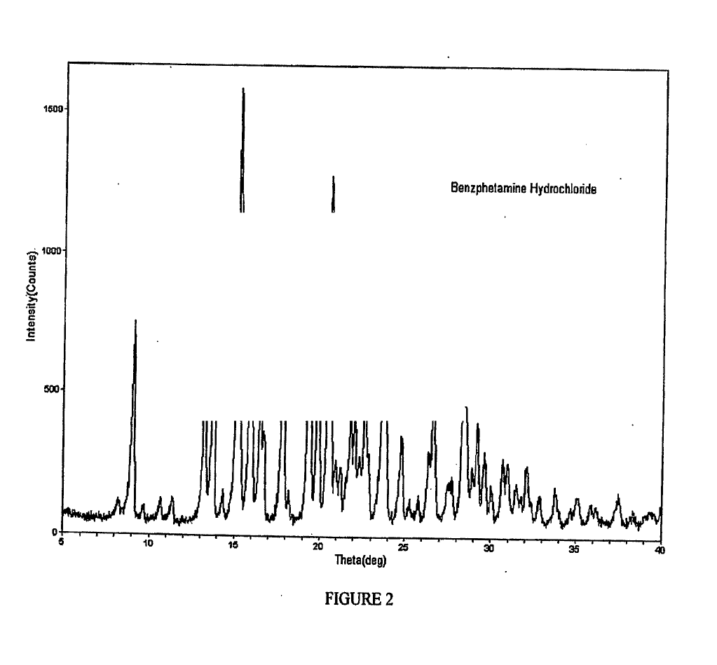

The PXRD pattern of benzphetamine hydrochloride is depicted in FIG. 2. Benzphetamine hydrochloride may be characterized by the PXRD characteristics set forth in Table 1.

TABLE 1

| Scattering Angle | |
|---|---|
| (degrees 2θ) | d-Spacing (\acute{Å}) |
| 8.3 | 10.62 |
| 9.2 | 9.62 |
| 9.8 | 9.00 |
| 10.8 | 8.22 |
| 11.4 | 7.73 |
| 13.4 | 6.62 |
| 13.9 | 6.38 |
| 14.5 | 6.12 |
| 15.3 | 5.77 |
| 16.1 | 5.51 |
| 16.6 | 5.32 |
| 17.9 | 4.94 |
| 19.5 | 4.54 |
| 20.0 | 4.43 |
| 20.7 | 4.29 |
| 21.1 | 4.22 |
| 21.3 | 4.16 |
| 22.2 | 4.00 |
| 22.8 | 3.90 |
| 23.9 | 3.72 |
| 24.9 | 3.58 |
| 25.4 | 3.50 |
| 25.9 | 3.44 |
| 26.5 | 3.36 |
| 26.8 | 3.33 |
| 27.9 | 3.20 |
| 28.6 | 3.12 |
| 29.3 | 3.05 |
| 29.7 | 3.00 |
| 30.1 | 2.96 |
| 30.8 | 2.90 |
| 31.1 | 2.87 |
| 31.6 | 2.83 |
| 32.2 | 2.77 |
| 32.9 | 2.72 |
| 33.9 | 2.64 |
| 35.2 | 2.55 |
| 36.0 | 2.50 |
| 36.3 | 2.48 |
| 37.6 | 2.39 |
| 38.4 | 2.34 |
| 39.4 | 2.28 |

The X-Ray diffractometer was Siemens D500 X-ray Diffractometer. The instrument utilized a Long Fine Focus X-ray Tube (Type: FL Cu 4KE) and a diffracted beam monochromator mounted in front of a scintillation detector. Samples were uniformly crushed (not ground) with a spatula edge, and dispersed on a quartz, zero-background holder. The experimental parameters are as follows:

Scan range—2.0 to 40.0 deg 2-theta

Display range—2.0 to 40.0 deg 2-theta

Step size—0.02 deg 2-theta

Scan time per step—1.0 seconds

Radiation source—copper Kα (1.5406 Å)

X-ray tube power—40 kV/30 mA

In one embodiment of the invention, crude benzphetamine hydrochloride is purified and crystallized by dissolving it into a solvent system, refluxing, and cooling. In this embodiment, the solvent system preferably comprises an organic solvent and an organic modifier. Crude benzphetamine hydrochloride is preferably insoluble in the organic solvent. Preferred organic solvents include ethyl acetate, propyl acetate, butyl acetate, toluene, heptane, acetonitrile, acetone, methylene chloride, chloroform, and mixtures thereof, with ethyl acetate currently preferred. The preferred organic solvents are miscible with alcohol. The solvent system also comprises an organic modifier which renders crude benzphetamine hydrochloride soluble in the solvent system. Preferred organic modifiers are alcohols, and the organic modifier may comprise methanol, ethanol, isopropanol, n-butanol, n-propanol, isobutanol, and combinations thereof, with n-butanol currently preferred. Preferably, the solvent system components and concentrations are selected such that crude benzphetamine has a solubility of at most about 5 g/L to about 15 g/L in the solvent system at room temperature, and at least about 30 g/L to 60 g/L (or more) at the boiling point. An exemplary solvent system which achieves this solubility is ethyl acetate modified with n-butanol in which between about 5 mL to 20 mL of ethyl acetate is used per gram of crude benzphetamine, preferably between about 10 mL to about 17.5 mL of ethyl acetate is used per grain of crude benzphetamine hydrochloride, and the solvent system comprises between about 5% to about 10% n-butanol by weight. More preferably, the liquid medium comprises about 10 mL of ethyl acetate per gram of crude benzphetamine hydrochloride and about 7.5% n-butanol by weight.

The crude benzphetamine hydrochloride solution is then heated to at least about 65° C., preferably between about 65° C. to about 85° C., more preferably between about 75° C. to about 80° C. and refluxed at about 75° C. to about 80° C., preferably for about 60 minutes. The solution can be stirred during heating and reflux.

After reflux, the solution is cooled to between about 5° C. and about 15° C., preferably to about 5° C. Cooling from the reflux temperature to ambient temperature may proceed by, for example, removing the heat source, but to reach temperatures between about 5° C. and about 15° C., the solution may be placed in an ice bath or cooled with a recirculating chiller. Cooling is preferably controlled such that the cooling rate is slow in order to produce crystals that have a mean particle size of approximately 100 to 400 microns. The cooling time from the reflux temperature to the final temperature is preferably between about 90 minutes to about 360 minutes. More preferably, the cooling time is between about 120 minutes and about 360 minutes. During cooling, the solution can be stirred. Once the solution reaches the final temperature, the solution is stirred at that temperature for at least about 60 minutes to obtain the highest possible yield. Preferably, the solution is stirred between about 60 minutes and about 120 minutes.

The process of refluxing the solution and slow, controlled cooling causes the formation of high purity benzphetamine hydrochloride crystals in high yield. The crystals may be isolated by filtering and washing with cold solvent in which the crystals are insoluble, preferably ice cold ethyl acetate, or by centrifugation. Refluxing and cooling according to the above described process in an ethyl acetate/n-butanol liquid medium, for example, results in crystallized benzphetamine in purity from about 95% to about 100% in about 60 to about 80% yield from the starting material. The final product contains no more than 0.15% of any single impurity and residual solvents are removed such that they are present at levels below permitted safety standards.

Advantageously, the purification and crystallization processes described above yield a higher purity, drier crystal product which may be reliably recrystallized to yield a very high purity recrystallized product with higher yield than known processes which do not utilize the purification and crystallization processes of the present invention.

Recrystallization involves re-dissolving the purified and crystallized benzphetamine hydrochloride in a suitable solvent system and slowly growing very high purity crystals by a variety of methods. Suitable solvent systems comprise at least one organic or inorganic solvent in which benzphetamine hydrochloride is soluble. Desirable solubility is at least 25 grams per liter at elevated temperature (boiling) and no more than about 5 grams per liter at cold (0 Celsius) temperatures. In some recrystallization processes, the solvent system may comprise inorganic solvent(s). Preferably, however, the solvent system comprises organic solvent(s) which are protic and volatile. In one embodiment, the organic solvents have a low boiling point (below 100° C.) to facilitate drying, low toxicity, a high degree of selectivity between benzphetamine hydrochloride and its impurities, and an absence of an azeotrope if a mixture of more than one solvent is used (in order to facilitate recovering or recycling the solvents.) Accordingly, suitable organic solvents include ethyl acetate, methanol, ethanol, isopropanol, N-butanol, acetone, acetonitrile, methylene chloride, and chloroform. Preferred organic solvents include ethyl acetate, acetone, and acetonitrile. In some recrystallization processes, the solvent system comprises two or more organic solvents. Preferable solvent systems with two or more organic solvents include N-butanol/ethyl acetate and methanol/ethyl acetate.

Recrystallization preferably occurs by slow growth of benzphetamine hydrochloride crystals from the organic solvent. This can be achieved, for example, by vapor diffusion or slow evaporation. Vapor diffusion involves the dissolution of benzphetamine crystals in a suitable liquid solvent and placing the liquid solvent in an atmosphere comprising a vapor. Preferably, the liquid solvent is an organic solvent such as isopropanol. Preferably, the atmosphere comprises a volatile organic molecule, such as ethyl acetate. In an exemplary isopropanol/ethyl acetate vapor diffusion system, about 185 mg of benzphetamine hydrochloride can be dissolved in approximately 2 mL of isopropanol in a small vial. The small vial can be placed into a 50 mL vessel, which can contain about 5 to about 6 mL of ethyl acetate. The vessel is then sealed.

Alternatively, recrystallization may occur by slow evaporation. Slow evaporation involves the dissolution of benzphetamine crystals in a suitable liquid solvent and allowing the liquid solvent to evaporate over the course of an extended period of time. Suitable solvents for crystal growth by slow evaporation include acetonitrile, water, acetone, methanol, ethanol, isopropanol, n-butanol, ethyl acetate, methylene chloride, and chloroform. Currently preferred solvents include acetonitrile and acetone. In some recrystallizations, the liquid solvent may comprise two or more solvents. An exemplary two-solvent system is ethyl acetate/methanol. Suitable solvents are capable of dissolving benzphetamine and are preferably volatile. Highly volatile solvents are not preferred however because rapid evaporation may result in amorphous material. Preferably, the benzphetamine crystals to be recrystallized are dissolved in the liquid solvent to saturated or near-saturated concentrations. For example, where acetonitrile is the solvent, between about 5 mg and about 10 mg benzphetamine is dissolved per 1 mL of acetonitrile solvent to reach near saturated solution. For acetone, between about 5 mg and about 10 mg benzphetamine is dissolved per 1 mL of acetone solvent to reach near saturation. For the two-solvent ethyl acetate/methanol system, between about 5 mg and about 10 mg benzphetamine is dissolved per 1 mL of liquid solvent to reach near saturation. The slow evaporation of the liquid solvent yields a supersaturated solution from which crystals consistent with benzphetamine hydrochloride of FIGS. 1 and 2 may form and grow with very high purity and high yield.

The high purity benzphetamine hydrochloride crystals of the present invention have sufficient purity for their intended pharmaceutical purpose. As such, benzphetamine crystals which were purified according to the processes of the present invention may be incorporated into pharmaceutical preparations.

### EXAMPLES

The following examples further illustrate the present invention.

### Example 1

For crystallization, ethyl acetate (49 mL) was added to benzphetamine hydrochloride oil (27.38 g), and the mixture was refluxed. The oil would not completely dissolve. After removal from heat, white crystals formed. The resin and crystals from ethyl acetate reflux were recrystallized using ethyl acetate (102.1 g) and isopropanol (17.29 g). Additional ethyl acetate (131.7 g) failed to yield crystals. So, the solution was stirred and cooled and crystals (4.59 g, first crop) formed after several minutes. The crystals were filtered and washed with ethyl acetate (50 mL) and dried in a vacuum oven. Overnight, an additional crop of crystals (8.09 g, second crop) formed in the filtrate. A sample of this second crop was taken for analysis.

The first crop of crystals had a melting point range from 153.9 to 155.5° C. Analysis showed that these crystals were 96.3% benzphetamine hydrochloride The second crop of crystals was dried in oven at 62° C. leaving 7.83 g of off-white solid having a melting point range between 154 and 156.3° C.

### Example 2

Crude benzphetamine hydrochloride (three samples of 2.00 grams each for crystallization from ethyl acetate/methanol, ethyl acetate/ethanol, and ethyl acetate/isopropanol and 0.93 grams for crystallization from ethyl acetate/n-butanol) was added to ethyl acetate (50 mL each for crystallization from ethyl acetate/methanol, ethyl acetate/ethanol, and ethyl acetate/isopropanol and 25 mL for crystallization from ethyl acetate/n-butanol) and heated to a very gentle boil in a water bath. Into each mixture was added enough organic modifier to dissolve the solids. In this experiment, the organic modifiers used were isopropanol, methanol, ethanol, and n-butanol. The four batches were removed from heat and allowed to cool to ambient temperature, and then cooled in an ice bath at 5° C. for 30 minutes. The slurries were filtered in a 4.25 cm Buchner funnel with Whatman No. 40 filter paper. The filter cakes were washed with ice cold ethyl acetate (7 mL). The samples were tested by HPLC, and the results are shown in Table 2.

TABLE 2

| | Alc. | | |
|---|---|---|---|
| | Vol. | Yield | Benzphetamine |
| Experiment | (mL) | (g) | Area % |
| Crude | N/A | N/A | 87.17 |
| Isopropanol#1 | 2.5 | 1.16 | 97.50 |
| Isopropanol#2 | | 0.02 | 3.52 |
| Isopropanol#3 | | 0.02 | 3.87 |
| Ethanol | 1.5 | 1.42 | 94.34 |
| Methanol | 1.00 | 1.36 | 95.03 |
| n-Butanol | 0.75 | 0.57 | 97.12 |

It was observed that the ethyl acetate/isopropanol filtrate continued to produce solids. Isopropanol #2 represents the content of the solids isolated one day after the initial experiment, and Isopropanol #3 represents the content of the solids isolated two days later. These samples contained almost all impurity.

The product of the n-butanol crystallization was purer than the products crystallized from methanol or ethanol. The yield from n-butanol crystallization was 61.3% (0.57 grams product from 0.93 grams crude). This exceeded the yield from isopropanol (58%) but was less than the yield from ethanol (71%) and methanol (68%.) The concentration of benzphetamine in the filtrate was 7.8 mg/mL, 6.07 mg/mL, 6.67 mg/mL, and 4.29 mg/mL for isopropanol, ethanol, methanol, and n-butanol crystallizations, respectively.

The above experiments were repeated (except for ethanol) and the results are summarized below in Table 3.

TABLE 3

| | Alc. Vol. | Yield | Benzphetamine |
|---|---|---|---|
| Experiment | (mL) | (g) | Area % |
| Crude | N/A | N/A | 96.36 |
| isopropanol | 2.5 | 1.56 | 99.08 |
| methanol | 0.75 | 1.61 | 99.25 |
| n-butanol | 2.25 | 1.53 | 99.42 |

The benzphetamine crystallized in ethyl acetate/n-butanol was purer than the material crystallized from other alcohols. In addition, particle-size characterization by Scanning Electron Microscopy (SEM) showed that the product crystallized using n-butanol had a better defined rod-like crystal morphology than product made with the other alcohols (although methanol gave very similar crystals) and toluene (plate-like crystal morphology). Without being bound by theory, it is thought that the rod-like crystal form produces a more uniform blend during formulation than plate-like crystals.

### Example 3

The following experiments were run according to the process set forth in Example 2:

TABLE 4

| Run Order/ | EtOAc Usage | N - butyl alcohol | Cooling Time |
|---|---|---|---|
| Notebook/Page | (ml/g) | Concentration (wt. %) | (minutes) |
| 1 discard | 10 | 15 | 150 |
| 2 | 25 | 10 | 90 |
| 3 | 25 | 5 | 90 |
| 4 | 17.5 | 7.5 | 120 |
| 5 | 25 | 5 | 150 |
| 6 | 10 | 5 | 90 |
| 7 | 10 | 10 | 90 |
| 8 | 10 | 5 | 150 |
| 9 | 25 | 10 | 150 |
| 1 rerun | 10 | 10 | 150 |

Results of the experiments are presented in Table 5. This table presents the percent yield of crystallized benzphetamine HCl and the area percent purity.

TABLE 5

| Experiment | Yield (%) | Area % |
|---|---|---|
| Crude | | 97.04 |
| 1 | 72.98 | 98.92 |
| 2 | 0.00 | N/A |
| 3 | 74.09 | 98.82 |
| 4 | 67.87 | 98.87 |
| 5 | 71.50 | 98.81 |
| 6 | 91.19 | 98.65 |
| 7 | 71.99 | 98.89 |
| 8 | 89.12 | 98.71 |
| 9 | 3.37 | 99.10 |

These results indicate that the yield was sensitive to the ethyl acetate usage and concentration of n-butanol. The yield improved as both were reduced. The cooling rate had no effect upon yield and a negligible effect upon purity.

The time the batch was stirred after reaching the final temperature was also investigated and found that a slight loss in yield results if the batch was stirred for less than 1 hour at the final temperature.

Further, particle size analysis was performed on these experiment samples by laser diffraction. It was found that finer particle sizes in stirred samples may be caused by using a low (5%) concentration of alcohol, and that coarse particle sizes in sonicated samples may be caused by a high (10%) concentration of alcohol. Without being bound to a particular theory it is thought that low concentrations of alcohol leads to less agglomeration of individual benzphetamine hydrochloride crystals, and that a high concentration leads to strong agglomerations or aggregations that do not disperse when exposed to ultrasound.

### Example 4

Crystallized benzphetamine hydrochloride was subjected to vapor diffusion recrystallization and analyzed by PXRD analysis. Benzphetamine hydrochloride (184.79 mg) was dissolved in isopropanol (2 mL) in a 10 mL vial. The vial was placed in a jar containing ethyl acetate (5 to 6 mL) and sealed. The slow vapor diffusion of ethyl acetate into the vial over a period of weeks produced crystals consistent with the PXRD pattern of benzphetamine hydrochloride of FIG. 2.

### Example 5

Crystallized benzphetamine hydrochloride was subjected to slow evaporation recrystallization from several solvents and analyzed by PXRD analysis.

Benzphetamine hydrochloride was added to saturated/near saturated solvent systems (1 mL each of acetonitrile, water, acetone, methanol, ethanol, isopropanol, n-butanol, ethyl acetate/methanol, methylene chloride, and chloroform). The resulting solutions were placed in small vials and set aside at room temperature in a nitrogen purged desiccator to allow for crystal growth.

Crystals obtained by slow evaporation were uniformly crushed with a spatula edge and dispersed on quartz zero-background holders. The powders were subjected to PXRD analysis. The results are summarized in Table 6.

TABLE 6

| Solvent | Powder Morphology |
|---|---|
| Acetonitrile | Crystals |
| Acetone | Crystals |
| Ethyl acetate/methanol | Crystals |

The powder patterns obtained from acetonitrile, acetone, and ethyl acetate/methanol grown crystals were consistent with the presence of benzphetamine hydrochloride of FIG. 2.

In view of the above, it will be seen that the several objects of the invention are achieved and other advantageous results attained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

All patents, applications, and other references cited herein are incorporated herein by reference in their entirety.

As various changes could be made in the above without departing from the scope of the invention, it is intended that all matter contained in the above description and shown in the accompanying drawings shall be interpreted as illustrative and not in a limiting sense.

What is claimed is:

1. A highly pure crystalline form of benzphetamine hydrochloride.

2. The crystalline form of benzphetamine hydrochloride of claim 1 having a purity of at least about 95% to about 100% by weight.

3. The crystalline form of benzphetamine hydrochloride of claim 1 having no more than 0.15% of any single impurity.

4. The crystalline form of benzphetamine hydrochloride of claim 1 having a melting point between about 151° C. and about 158° C.

5. The crystalline form of benzphetamine hydrochloride of claim 1 having a melting point between about 155° C. and about 157° C.

6. The crystalline form of benzphetamine hydrochloride of claim 1 having rod-like crystal morphology.

7. The crystalline form of benzphetamine hydrochloride of claim 1 having plate-like crystal morphology.

8. A process for preparing a highly pure crystalline form of benzphetamine hydrochloride comprising:
a) dissolving benzphetamine hydrochloride in a solvent system comprising an organic solvent and an organic modifier to form a solution comprising benzphetamine hydrochloride, the organic solvent, and the organic modifier;
b) heating the solution to a temperature sufficient to dissolve the benzphetamine hydrochloride in the solvent system; and
c) cooling the solution to a temperature sufficient to precipitate benzphetamine hydrochloride into the highly pure crystalline form of benzphetamine hydrochloride.

9. The process of claim 8 wherein the organic solvent is miscible with alcohol.

10. The process of claim 8 wherein the organic solvent is selected from the group consisting of isopropanol, ethanol, methanol, ethyl acetate, propyl acetate, butyl acetate, toluene, heptane, acetonitrile, acetone, methylene chloride, chloroform, and mixtures thereof.

11. The process of claim 8 wherein the organic solvent is ethyl acetate.

12. The process of claim 11 wherein between about 5 mL to about 20 mL of ethyl acetate is used per gram of benzphetamine hydrochloride.

13. The process of claim 11 wherein between about 10 mL to about 17.5 mL of ethyl acetate is used per gram of benzphetamine hydrochloride.

14. The process of claim 11 wherein the organic modifier is selected from the group consisting of methanol, ethanol, isopropanol, n-butanol, n-propanol, isobutanol, t-butyl alcohol and combinations thereof.

15. The process of claim 8 wherein the modifier is n-butanol.

16. The process of claim 15 wherein the solvent system comprises n-butanol in a concentration between about 5% and about 10%.

17. The process of claim 15 wherein the solvent system comprises n-butanol in a concentration of about 7.5%.

18. The process of claim 11 wherein heating the solution comprises heating to a temperature of at least about 65° C.

19. The process of claim 11 wherein heating the solution further comprises the step of refluxing the solution at a temperature between about 65° C. and about 85° C. between about 15 minutes and about 60 minutes.

20. The process of claim 11 wherein cooling the solution comprises cooling to a temperature between about 5° C. and about 15° C. occurs over a cooling time between about 90 minutes to about 360 minutes.

21. The process of claim 11 wherein cooling the solution comprises dissolving the benzphetamine hydrochloride at a temperature between about 5° C. and about 15° C. for at least about 60 minutes.

22. The process of claim 11 further comprising the step of isolating the crystalline form of benzphetamine hydrochloride by filtration, distillation, and centrifugation.

23. The process of claim 11 further comprising the step of recrystallizing the crystalline form of benzphetamine hydrochloride by vapor diffusion or slow evaporation.

24. The process of claim 23, wherein the slow evaporation is conducted in solvent systems selected from the group consisting of acetonitrile, acetone, and ethyl acetate/methanol.

25. A process for preparing a highly pure crystalline form of benzphetamine hydrochloride comprising:
a) dispersing benzphetamine hydrochloride in a liquid medium in which benzphetamine hydrochloride is essentially insoluble to form a biphasic mixture comprising benzphetamine hydrochloride and the liquid medium;
b) heating the biphasic mixture to a temperature between about 40° C. and about 95° C.; and
c) cooling the biphasic mixture to precipitate the highly pure crystalline form of benzphetamine hydrochloride.

26. The process of claim 25 wherein the liquid medium is selected from the group consisting of toluene, ethyl acetate, and xylenes.

27. The process of claim 25 wherein the biphasic mixture is heated to a temperature between about 40° C. and about 95° C.

28. The process of claim 25 further comprising the step of agitating the biphasic mixture.

29. The process of claim 28 wherein agitating the mixture comprises stirring the mixture at about 500 to about 1200 rpm.

30. The process of claim 28 wherein agitating the mixture comprises stirring the mixture at about 120 rpm with a stirrer having a paddle with a 2.5 ft radius.

31. The process of claim 25 further comprising the step of isolating the highly pure crystalline form of benzphetamine hydrochloride by filtration or centrifugation.

32. The process of claim 25 further comprising the step of recrystallizing the purified solid form of the crystalline form of benzphetamine hydrochloride.
